(19) United States
(12) Patent Application Publication
(10) Pub. No.: US 2004/0023903 A1
(43) Pub. Date: Feb. 5, 2004

(54) Single-stranded end-capped oligonucleotide mediated targeted gene repair and modification and uses thereof

(76) Inventors: Brian Ronald Davis, Houston, TX (US); David Bruce Brown, League City, TX (US); N.D. Victor Carsrud, (US)

Correspondence Address: MERCHANT & GOULD PC, P.O. BOX 2903, MINNEAPOLIS, MN, 55402-0903, US

(21) Appl. No.: 09/767,775

(22) Filed: Aug. 14, 2001

Related U.S. Application Data
(63) Continuation-in-part of application No. 09/336,655, filed on Jun. 18, 1999, now abandoned.
Application No. 09/336,655 is a continuation-in-part of application No. PCT/US97/23781, filed on Dec. 20, 1997.

(30) Foreign Application Priority Data
Dec. 19, 1997 (EP) 97952611.8
Dec. 19, 1997 (AU) 56182/98
Dec. 19, 1997 (CA) 2,275,474
Dec. 19, 1997 (JP) 529047/1998

(51) Int. Cl. A61K 48/00

(52) U.S. Cl. 514/44

(57) ABSTRACT

The present invention relates to the field of transfer of small molecules of exogenous nucleic acid into living cells, and to improved methods for accomplishing same using the technique of single-stranded end-capped oligonucleotide gene repair. In some embodiments, the inventive method may be further described as providing incorporation of said materials into cells that can be made to exist in an adherent state in vitro. The invention also relates to the field of microinjecting said materials into living cells with improved cell viability for the injected cells. As well, the invention describes a method for improved genetic modification of endogenous sequences using co-delivery of accessory proteins and oligonucleotides to facilitate modification. The invention further relates to the field of gene therapy, using the technique of single-stranded end-capped oligonucleotide gene repair to correct genetic defects, as well as introducing specific mutations into genomic DNA for use in functional genomics. In some embodiments the invention may be used to introduce specific genetic mutations into selected genes of living cells for the purpose of generating transgenic mice, isogenic cell lines, primary cell types carrying a specific mutation, genetically modified plant cells, validation of gene function, and including, but not limited to, disease gene discovery.

The present application is a continuation in part of U.S. application Ser. No. 60/033,820 filed Dec. 20, 1996, which was followed by international application PCT/US/97/23781, filed Dec. 20, 1997, which was followed by U.S. application Ser. No. 09/336,655, filed Apr. 19, 2000.

### BACKGROUND OF THE INVENTION

Gene therapy through targeted modification and/or correction of disease-causing mutations within the human genome provides one approach to treatment of genetic and acquired diseases. Generally, targeted gene modification as used in the description of the present invention may be described as the delivery of a small molecule of nucleic acid, that is highly homologous (similar) to a gene region of interest that contains a genetic mutation, with the introduced nucleic acid containing a native (wild-type), corrected or modified DNA sequence. According to this approach, the cell's normal molecular machinery (involving mismatch repair, homologous recombination, other processes, and combinations of processes) exchanges the mutant sequence region within the gene with a corrected (wild-type) sequence region, thereby, correcting or repairing the gene. These techniques can be used to create specific mutations or alterations within a specific gene of interest in functional genomics applications (i.e. target gene validation). In contrast, gene compensation, involving the delivery of corrective DNA (usually composed of the entire coding region of a gene and appropriate regulatory sequences), merely overrides or compensates for the defective gene. The defective gene still remains in the affected cells. Utilization of targeted gene modification technology bypasses a number of difficulties associated with gene compensation methodologies, especially viral-based gene compensation strategies.

A technique that would correct the defect itself using gene repair that maintains the corrected genetic material within its normal chromatin environment permits appropriate genetic regulation and expression in the cell. Indeed, targeted gene-modification technology is ideally suited for genetic diseases that result from well-defined, limited alterations in the DNA sequence, such as Cystic Fibrosis, Sickle Cell Anemia, and the Thalassemias. In addition, gene repair may be the only suitable genetic modification in situations where the mutant gene product exercises a dominant negative influence over the normal gene product. For example, expression of mutant collagen chains in the disease Osteogenesis Imperfecta cannot be obviated by over-expression of normal collagen. Thus, the introduction of an intact, healthy collagen gene via gene compensation strategies would not be expected to treat and/or cure the disease. However, correction of the mutant collagen genes in theory would provide therapeutic benefit.

Considerable investigation has been performed to develop technologies that allow for targeted modification of genes within the chromatin. Gene targeting using larger regions of isogenic DNA together with single or double drug selection, has been widely used for generating knock-out or knock-in mice. However, the frequencies of gene correction observed (10⁻⁵ or 1 in 100,000 cells) (0.00001%) are generally insufficient for therapeutic applications or high through-put screening applications. While suitably constructed adeno-associated virus (AAV) vectors are capable of targeted gene modification or gene repair (Piacibello, et al. (1997), Blood, 89:2644; Russell, et al. (1998), Nature Genetics, 18:325), the frequencies of repair reported are generally still too low for clinical application. Some classes of repair molecules have been described that report to provide correction frequencies sufficient for clinical benefit greater than those achieved by standard gene targeting methodologies. Goncz, et al. (1998), Human Molecular Genetics, 7:1913; Inoue, et al. (1999), Journal of Virology, 73:7376; Kunzelmann, et al. (1996), Gene Therapy, 3:859; Cole-Strauss, et al. (1996), Science, 273:1386; Kren, et al. (1999), Proc. Natl. Acad. Sci. U.S.A., 96:10349; Xiang, et al. (1997), Journal of Molecular Medicine, 75:829; Russell, et al. (1998), Nature Genetics, 18:325.

A unique gene modification method utilizing a construct composed of both RNA and DNA has shown reasonable evidence of gene repair. These molecules, referred to as chimeras, have a DNA strand complemented to an RNA/DNA strand connected by hairpin loops at both ends. These chimeras can elicit point mutations and single base insertion corrections when exposed to a cell-free extract at a rate that is significantly higher (several logs higher in fact) than with either single or double stranded, naked and unmodified DNA constructs alone (Cole-Strauss, et al, 1999). Later evidence examined the function of each of the structural components of the chimeric molecule and has identified the active strand of the chimera that acts as a template for gene correction as the strand composed purely of DNA. It has been proposed that the RNA/DNA strand of the chimera is responsible for facilitating recombinational complex formation, and that the hairpin loops at either end of the construct act as protective structures to degradation. Recently, these initial suggestions were substantiated by the use of a single-stranded, end-capped oligonucleotide to effect gene conversion in both animal and plant cell-free extracts which generate conversion rates ˜3-4 fold higher than that effected by the original chimeric molecules.

The primary limitation to the efficiency of DNA- or RNA/DNA-mediated gene correction is at least in part attributed to inefficient delivery of a sufficient number of repair molecules to the nuclei of target cells (Cole-Strauss (1996), Science, 273:1386). Hence, a need continues to exist in the art for more efficient approaches for incorporating nucleic acid sequences into a target population of cells. Such improvements would also provide superior molecular repair agents for use in the correction of genetic disease.

### SUMMARY OF THE INVENTION

The present invention relates generally to the field of genetic repair, modification, correction, and enhancement through the transfer of small molecules of exogenous nucleic acid molecules alone or together with other molecules into living cells, as well as to methods using the technique of single-stranded end-capped oligonucleotide gene repair to achieve genetic repair, modification, and enhancement with preserved cell viability and/or biologic function in the modified cell population. The invention further describes a method for improved genetic modification of endogenous sequences using co-delivery of accessory factors. These accessory factors may include by way of example and not by limitation, proteins, peptides, nucleic acids, and mixtures thereof. The invention further relates to the field of gene therapy, using the technique of single-stranded end-capped oligonucleotide gene repair to correct/repair genetic defects, as well as to the field of functional genomics where single-stranded end-capped oligonucleotides are used for introducing specific mutations into genomic DNA.

In some embodiments, the invention may be used to introduce specific genetic mutations into selected genes of living cells for the purpose of generating transgenic mice and isogenic cell lines. In other embodiments, the inventive method may be further described as providing incorporation of relatively small nucleic acid molecules, alone or together with other materials, into cells to create small, targeted mutation sites in a nucleic acid sequence of a cell. These mutated sequences may then also be used in functional genomics studies.

The present invention provides a non-viral microinjection-mediated delivery as an improved method for targeted gene modification, particularly since microinjection allows for direct delivery of specialized, small molecules of genetic material into the nucleus of a cell. Moreover, the methods outlined in the present invention are ideally suited for introduction of targeted gene modification molecules to stem cells, particularly blood stem cells, for the purposes of treatment of a variety of diseases via gene therapy. Furthermore, the methods outlined in the present invention are suited for introduction of targeted gene modification molecules to stem cell types. These stem cell types include, but are not limited to, hepatic, pancreatic, mesenchymal, and neuronal stem cells.

In the present invention, the relatively short length nucleic acid sequence that is used to repair a targeted mutant sequence may be further defined as having a length of from 1 to 400 nucleotide bases. This includes, but is not limited to, overall lengths of 25, 51, 68, 88, 98, 100, 150, 175, 200, 300, 350, or 400 nucleotides. This repair nucleic acid sequence may include the “repair” to a target mutant cell of one (1) to one hundred (100), or one (1) to fifty (50) mutated bases, or more particularly from one (1) to twenty (20) mutated bases, or even one (1) to ten (10) mutated bases, or five (5), two (2) or a single mutated base in the mutated sequence in the mutant cell. In some forms of the invention, 2 distinct nucleic acid modifications will be created in a targeted gene, where one modification will, in some embodiments, include the correction of a specific mutation responsible for a pathology. The second such modification will involve introduction of a modification at a restriction enzyme site for the purposes of detection of the targeted modification. In other embodiments of the invention the generation of 2 distinct modifications via targeted gene modification may involve the introduction of one such modification targeted at the sequence responsible for pathology and one such modification targeted at a site which may generate altered drug resistance or sensitivity for the purposes of selection of modified cells. In another embodiment of the invention, the single stranded end-capped oligonucleotides can be used to modify more than one point mutation, insertion, or deletion within the same gene, for example, a gene whose defect is the result of two point mutations. In these cases, either two single stranded end-capped oligonucleotides could be used, or a single, large oligo with two corrective regions could be used

Single-Stranded End-Capped Oligonucleotide Gene Repair

In some embodiments, the invention may be defined as directed to highly effective techniques for correcting genetic disease in animals at the molecular level of the gene. In other embodiments, the invention provides a method for repairing a gene or a part of a gene sequence in a manner that is both permanent and specific. Fairly small length, single stranded oligonucleotides capped with modified nucleotides are used to repair specific defects within a cell, such that the mutated form of the gene in the corrected cell is eliminated. In this regard, within the target population of cells, there would be present modified cells that did not include the unmodified forms of the sequence. In other embodiments, the invention provides a method for repairing one of the copies or alleles of a gene or a part of a gene sequence in a manner that is both permanent and specific. In this regard, the corrected cell would include one copy of the mutated form of the sequence and one copy of the corrected, repaired, wild-type, or modified form of the sequence.

In yet other embodiments, it is envisioned that the nucleic acid molecules, particularly the DNAs that will be used in the present invention, will be highly similar in DNA sequence to the gene region targeted for repair, and will include the corrected, normal wild-type sequence where the gene sequence causing the phenotypically observed disease in the animal was not the wild-type sequence. These small molecules of DNA are delivered to the nucleus as single stranded, “naked” oligonucleotide molecules. As used in the description of the present invention, the term “naked” oligonucleotide is defined as a sequence of nucleic acids which are not within the confines of a carrier molecule, liposome, virus or other carrier molecule, or complexed with a protein or other accessory molecule.

The term “foreign material,” as used in the description of the present invention, includes RNA, DNA (single or double stranded), duplex RNA and DNA, modified forms of RNA and DNA, modified/chemically modified duplex RNA and DNA, a peptide and nucleic acid molecule, a synthetic molecule such as a modified nucleic acid sequence useful to target nucleic acid molecules capable of doing repair together with the repair molecule sequence of interest, or a combination thereof.

Once delivered inside the nucleus of the cell, the DNAs find the target sequence, and then endogenous cellular molecular machinery recombines the correct sequence of DNA with the “target” defective sequence, thereby replacing the defect with the correct sequence. Alternatively, co-delivery of accessory factors may interact and work with introduced DNA and endogenous cellular machinery to bring about replacement/correction of the target sequence.

DNA/RNA chimeric molecules (chimeras) have been shown to correct transversions generating stop codons in plasmids encoding the kanamycin gene and tetracyclin resistance gene, indicating that these chimeric sequences can effect efficient correction (Cole-Strauss, 1999). It has been shown that delivery of a single stranded DNA similar to the DNA strand of the chimeric molecule, with such ends capped with modified nucleotides so as to provide degradative protection, is a more efficient and effective method for targeted gene correction than via previous chimeric oligonucleotide methods for gene repair (Gamper, et al, 2000).

Microinjection-Mediated Delivery:

Microinjection of macromolecules (e.g., antibodies, mRNA, DNA) into living cells has proven to be a powerful approach for studying the biology of cells at the molecular level. The microinjection method practiced according to the invention in some embodiments employs non-adherent cells and microinjection needles, the needles having an outer diameter of about 0.05 microns to about 0.5 microns, and a particularly defined flare region defined by a ratio D1 :D2, as measured from the tip to the widest width of the needle. (U.S. Ser. No. 09/336,655, specifically incorporated herein by reference for this purpose). The present methods in other applications provides for immobilization of a non-adherent cell onto a surface, followed by microinjection of the cell to include a desired foreign material. The invention further provides for the removal of genetically modified cells from a culture surface with minimal damage and/or very low loss of cell viability and/or biologic function of the cells.

### BRIEF DESCRIPTION OF THE DRAWINGS

FIGS. 1A-1C): Graphically describes blood stem cell differentiation in a normal healthy patient. 1A) The body contains specialized cells known as somatic stem cells which have the unique ability to not only duplicate themselves, but also to produce different cells; these different cell types may go on to adopt unique characteristics during the course of growth and development. Thus, a single stem cell type may ultimately give rise to several types of cells, which serve different specialized functions within the body. For example, the blood stem cell regenerates itself, but is also the ‘mother’ of all the types of cells making up the blood system, including platelets (responsible for clotting), the various white blood cells (responsible for immunity), as well as the red blood cells (responsible for transporting oxygen throughout the body). Correct, non-mutant genes within the normal, healthy cells produce the appropriate wild-type proteins specifically expressed by each cell type. 1B) In patients with Sickle Cell Disease, the mutant Sickle allele is present in all stem cells and hence in all progeny of the these cells. However, due to controlled differential expression, the gene coding for the mutant hemoglobin is expressed only in (red blood cell) RBC precursors and not in platelets or white blood cells. 1C) After modification of the mutant hemoglobin sequence in a Sickle Cell Patient's stem cells, the corrected gene will produce the normal protein which will be expressed in the patients RBCs.

FIG. 2: The present invention provides for gene modification of somatic stem cells that will provide therapeutic benefits for patients suffering inherited or acquired genetic diseases. The gene therapy is ex vivo, as all genetic modifications occur outside the body of the patient. Briefly, the appropriate stem cells would first be harvested from a patient's tissue sample, and then corrected using a non-viral gene modification protocol as described in the present invention. Molecular analysis would then be performed to identify (and expand, if necessary) the corrected cell population. The corrected cells would then be infused back to the patient.

FIGS. 3A, 3B and 3C: The technology described in the current application has application to functional genomics (3A), gene therapy (3B), and the field of transgenics (3C). A method of rapidly and efficiently changing specific sequences of DNA in cells or animals and observing the results is essential for gene therapy and functional studies.

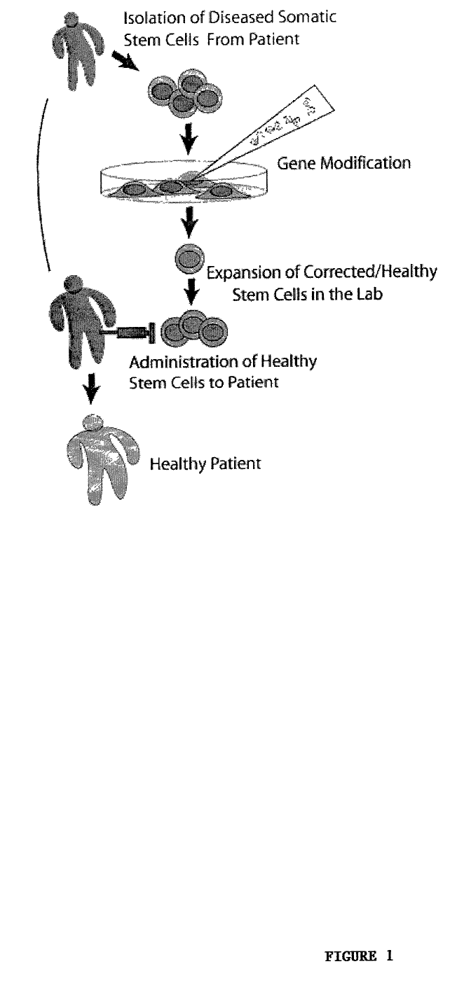

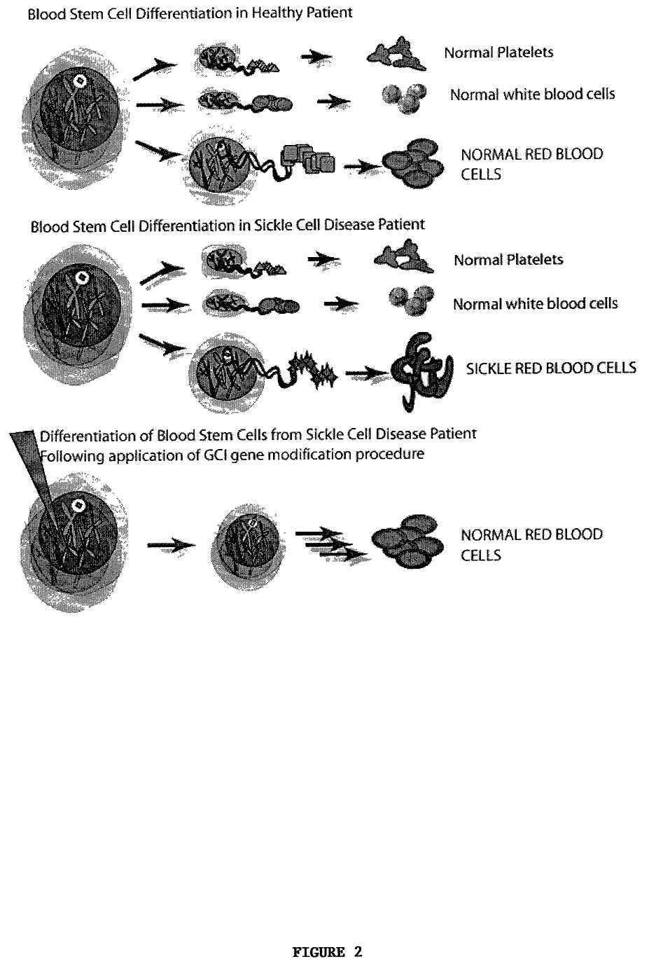

### DETAILED DESCRIPTION OF THE PREFERRED EMBODIMENT

The present specification, examples and data provide a complete description of the manufacture and use of the composition of the invention. Since many embodiments of the invention can be made without departing from the spirit and scope of the invention, the invention resides in the claims hereinafter appended.

The present invention provides a non-viral mediated method for the incorporation of nucleic acid molecules capable of repairing one or more genetic defects at the gene, mRNA, and/or protein level in an animal having a genetic defect. Through this method, reduction and/or elimination of the phenotypically detectable consequence of genetic disease in the animal can be accomplished. In particular embodiments, the invention relates to the microinjection-mediated delivery of relatively small pieces of single stranded nucleic acid, having a length of typically between 1 to 200 bases, and having a sequence of modified nucleic acids of variable number (about 10% to about 30% of the entire length of the sequence to be used in creating a modified population of target cells) on each end to cap the molecule. Upon the relatively efficient delivery of such nucleic acid molecules to a target population of mutant cells (i.e., diseased cells in need of genetic repair), a population of genetically modified cells that include a specific nucleotide change at a single or relatively small genomic locus can be provided, thus providing a correction of the defect in these cells. In other embodiments, the target population of cells contains phenotypically and genotypically normal cells which are to be genetically modified at a specific gene of interest.

The population of genetically modified cells provided according to the present invention can be used in a variety of applications, including: (a) functional genomics, and (b) to treat a physiological disorder. In this regard, the techniques disclosed herein may be used in gene therapy. In another aspect, the invention provides for a preparation of cells having a cell population enriched for genetically modified cells. Such preparations may also be administered parenterally to a patient suffering from a gene therapy-responsive physiological disorder, wherein the genetically modified cell and its progeny may express a therapeutic agent. It is anticipated that this would provide a treatment for a patient's physiological disorder. Genetically modified human stem cells (hSCs) prepared according to the methods of the present invention can be employed for gene therapy applications once said modified hSCs have been delivered to humans for long-term reconstitution.

According to the present invention, hematopoietic stem cells that have been modified by microinjection of foreign material can be used to treat a variety of physiological disorders such as, by way of example and without limitation, AIDS, cancer, thalassemia, anemia, sickle cell anemia, adenosine deaminase deficiency, Fanconi Anemia, Gaucher disease, Hurler Syndrome, immune deficiencies, and metabolic diseases.

The physiological disorders contemplated within the invention will be responsive to gene therapy. By “responsive to gene therapy” is meant that a patient suffering from such disorder will enjoy a therapeutic or clinical benefit such as improved symptomology or prognosis.

The present invention also contemplates the microinjection-mediated delivery of single-stranded end-capped oligonucleotide molecules with accessory molecules including, but not limited to, integration proteins, molecules that enhance homologous recombination and molecules that enhance the mismatch repair pathway. Microinjection-mediated delivery directly to the nucleus of cells would be more efficient than delivery of same with retroviruses, liposomes, dendrimers, or other indirect methods of delivery.

Microinjection allows for the delivery of therapeutic DNA directly to the nucleus of an individual cell, bypassing some of the limitations of traditional, virus-based methods. The ability to deliver novel gene modification molecules makes the technology attractive for application in not only somatic stem cell gene therapy, but also complementary commercial fields such as functional genomics, target gene validation, proteomics, and transgenics. As such, the present invention defines a platform expected to impact disease treatment, biotechnology research, and functional genomics studies.

The primary human blood cells that are the progeny of modified hSCs and which can be used in the present invention include, by way of example, leukocytes, granulocytes, monocytes, macrophages, lymphocytes, and erythroblasts. For example, stem-cells from thalassemic or sickle cell anemia patients that are genetically modified with the appropriate hemoglobin gene may give rise to genetically corrected red blood cells.

Gene Therapy

The present invention provides for gene modification of somatic stem cells that will provide therapeutic benefits for patients suffering inherited or acquired genetic diseases. The gene therapy is ex vivo, as all genetic modifications occur outside the body of the patient. Briefly, the appropriate stem cells will be purified from a patient's tissue sample, the genetic defect will be corrected using the non-viral gene modification protocols described in the present invention, molecular analysis will be performed to identify (and expand if necessary) the corrected cell population, then the modified cells will be returned to the patient. To illustrate the concept, a patient suffering from sickle cell anemia, a blood disorder resulting from a small change in the sequence of a globin gene responsible for producing hemoglobin, a key molecule in red blood cells responsible for transporting oxygen throughout the body, may be treated using the present invention methods. The mutation in the globin gene results in red blood cells with an a typical ‘sickle’ shape. Sickle cell patients often suffer from increased heart trouble and general medical complications resulting from the inability of the abnormally shaped red blood cells to deliver appropriate amounts of oxygen throughout the body. Red blood cells have a short life span (approximately 120 days), and must be continually generated. For gene therapy, bone marrow/tissue containing blood stem cells would be isolated from the sickle cell patient at a hospital or clinic. The stem cells would be purified, and molecules designed to repair the defective globin gene would be delivered to the cells using the herein-described non-viral method. The presence of the corrected gene would be confirmed by molecular analysis, and the corrected cells administered back to the original patient. Once returned to the patient, the corrected stem cells would migrate back to the bone marrow. Established there, the corrected stem cells multiply, continually producing cells, ultimately resulting in red blood cells with the correct hemoglobin, restoring a normal level of oxygen transport in the blood.

Functional Genomics

Functional genomics refers to ascribing a specific, known DNA sequence to a particular function, and/or linking a specific change in a DNA sequence with a particular pathological outcome or disease. DNA sequences can be classified by three general categories: sequences that are the ‘blueprint’ for a protein; sequences that regulate the building of the protein; and sequences that have not been attributed to a particular function. In the future, great emphasis will be placed on determining the function of the 80,000-120,000 human genes identified through the human genome project. In this regard, the present inventive methods may be used to perform functional genomics studies directed at determining the function of genes whose function has not yet been defined. This may involve the introduction of either knock-out mutations (to evaluate the consequence of eliminating expression of a particular gene), specific genetic mutations (to evaluate, for example, the role of particular gene sequences in the functioning of the expressed protein), or specific naturally occurring genetic polymorphisms) to evaluate, for example, the role of specific single nucleic acid polymorphisms [SNPs] in predisposition to a particular disease).

### EXAMPLE 1

### Delivery of Single-Stranded End-Capped Oligonucleotide Molecules to hHSCs via Microinjection

The present example demonstrates the utility of the invention for providing an effective mechanism for genetically modifying undifferentiated cell types. By way of example, such cells include, but are not limited to, human blood stem/progenitor cells. In some embodiments, the present invention will be used to microinject single-stranded end-capped oligonucleotide molecules into the nucleus of human hematopoietic stem cells (HSCs). The utility of delivering single-stranded end-capped oligonucleotide molecules for a therapeutic effect by recombination with a targeted site having a defined effect on the genome is therefore demonstrated. Such a gene repair effect is similar to that seen in the cell-free extract demonstrated by Gamper, et al, (2000), in which kanamycin resistance was repaired on introduced pK^{s}m4021 plasmids in the extract via the single stranded oligonucleotides for an observable resistance in the resultant colonies.

HSCs may be isolated from either patient or donor bone marrow. These cells will be further purified from a population of recovered cells by selection for the presence or absence of cellular markers in order to provide cells with expression such as, but not exclusive to, CD38⁻/lin⁻/CD34⁺/KDR⁺. Approximately 1-100×10³ cells of this phenotype will be then temporarily immobilized on plates coated with the fibronectin derivative, retronectin or other adhesive molecule as described in the parent patent application, U.S. Ser. No. 09/336,655.

Once temporarily immobilized, the injections will be performed using needles defined as having an outer tip diameter between 0.01 and 0.5 μm (D1) and a second diameter (D2) a distance (L) away along the needle shaft defined as the flare region. The ratio of D2/D1 shall be known as the flare ratio, and shall vary amongst these needles between 1:1.8 and 1:3. The needles shall be able to consistently microinject hHSCs with viabilities of 65% and greater as described in the parent patent application, U.S. Ser. No. 09/336,655.

The formulation that is to be incorporated (such as by injection) into the cell may in some embodiments comprise single-stranded end-capped oligonucleotides and a solution of about 114 mM KCl and about 3 mM KPO₄ (pH 7.4). In other embodiments the solution may comprise buffers other than that listed above which do not interfere with the Single-stranded end-capped oligonucleotides' activity or cell viability and/or biologic function. The sample may also comprise additional accessory molecules. These accessory molecules most frequently will be molecules that will increase the efficiency of treatment to the hHSCs.

Microinjection, one technique that may be used in the practice of the invention for introducing materials into a cell, will then be performed upon an effective number of cells. An effective number of injected cells will be determined based on the observed effect of the single-stranded end-capped oligonucleotide constructs defined in prior studies. Typically, the constructs of the invention will contain target sequencing to a specific genetic defect within the cellular genome. Single-stranded end-capped oligonucleotides will be introduced at concentrations including, but not limited to, the range between 1000 and 5000 copies of the “corrective” small single stranded end-capped DNA molecule per femtoliter (fL). Each hHSC will receive a volume of injected material including, but not limited to, the range between 0.5 and 2 fL into its nucleus upon successful injection.

After injection, the cells will be detached, for example as described in U.S. Ser. No. 09/336,655, hereby specifically incorporated herein by reference. The treated/corrected cells will either be directly re-introduced into the patient or will first be expanded to sufficient numbers in laboratory culture conditions prior to re-introduction into the patient. The corrected cells may be expanded using techniques well known to those of skill in the art.

Typical methods for expanding cells are described in Piacibello, et al. (1997), Blood, 89:2644, which article is specifically incorporated here by reference. This technique may be used in order to foster the growth of a number of cells sufficient to provide the therapeutic or other effect desired in a target animal. By way of example, a sufficient number of expanded cells would comprise about 40,000 bone marrow-derived hHSCs of the phenotype CD38⁻lin⁻/CD34⁺/KDR⁺ (Will, A. M. (1999), Archives of Disease in Childhood, 80:3; Theilgaard-Monch, K., et al. (1999), European Journal of Haemotology, 62:174; Kelly, P., et al. (1997), Journal of Pediatrics, 130:695; Lu, L., et al. (1996), Critical Reviews in Oncology-Hematology, 22:61; Wang, J. C., et al. (1997), Blood, 89:3919; Fritsch, G., et al. (2000), Bone Marrow Transplantation, 17:169; Kogler, G., et al. (1999), Klinische Padiatrie, 211:224; Gluckman, E. (1996), Bone Marrow Transplantation, 18:166; Christianson, S. W., et al. (1997), Journal of Immunology, 158:3578; Bandyopadhyay, P., et al. (1999), Journal of Biological Chemistry, 274:10163) corrected cells having the corrected β-globin gene sequence would be used in the treatment of a patient having sickle cell anemia.

The corrected cells may be further treated with molecular factors in order to help select for more mature cells of a particular cellular lineage of differentiation, such as but not limited to, various cytokines. By way of example, the cells can be exposed to a mixture of GM-CSF, IL-3, and EPO in order to select for a population enriched in an erythrocyte lineage.

Upon sufficient expansion, the treated stem cells will then be introduced/reintroduced into the patient for the observed clinical effect mediated by the introduced single-stranded end-capped oligonucleotides.

### EXAMPLE 2

### Utilization of Various End Capping Modified Nucleotides for the Purpose of Degradative Protection for the Small Single Stranded End-Capped Oligonucleotides

The present example demonstrates some, but not all of, the various modified forms of nucleotides that can be added to either end of the single stranded DNA oligonucleotide used in this technology.

It has been widely accepted that a primary limitation to the efficacy of the introduction of conventional single or double stranded DNA molecules in gene repair is the rapid degradation of the molecule. Exposed, free DNA ends are susceptible to attack by exo-nucleases native to every cell. The level and activity of these exonucleases, some involved in protection of the cell to viral infection, varies from cell to cell based upon cell type and environmental signaling. However, just as these exonucleases degrade invasive viral nucleotide sequences, so they also rapidly degrade introduced therapeutic sequences. Gamper et al (2000) demonstrated that by protection of the oligonucleotide from exonucleolytic degradation can increase the rate of gene repair by up to 4 fold, dependent upon the precise form and number of the protective modification. This was further confirmed therein by electrophoretic analysis of the degradation of protected vs. unprotected DNA oligonucleotides in the experimental cell-free extract.

Stability against these exonucleases is accomplished in a variety of fashions. Structural protection, for example, is accomplished in therapeutic RNA/DNA chimeras by the design of T-rich hair-pin loops, and is accomplished in single stranded oligonucleotides by the addition of modified nucleotides to either end (Gamper, et al, 2000). Such end cap nucleotides are further shown to include, but are not limited to, phosphorothioate linkages between nucleotides. There is shown a distinct relationship between the length and number of the modification to either end of the oligonucleotide, and the length of the intervening, repairing DNA sequence. For a single stranded molecule of 25 nucleotides of 25 bases, for example, phosphorothioate linkages of between 3 and 6 nucleotides on each end is most effective at stabilization for mediation of the observed gene repair. Decreasing effectiveness is observed with increasing numbers of modified bases on either end of a molecule with a total length of 25 nucleotides, suggesting inhibition of the desired repair effect with a shorter intervening sequence. This “region of repair” of unmodified bases necessary for effectiveness is hypothesized to be larger than the targeted repair site alone (Gamper, et al, 2000).

Other protective modifications are here hypothesized to include a backbone of methylphosphonate, phosphoramididate, morpholino peptide linkages, or containing different 2′-halo, 2′-alkyl, or 2′-alkoxylalklyl sugars. Gamper et al (2000) demonstrated that other modifications can impart such protection, such as their use of 2′-O-methyl ribonucleotides, though such protection was determined to be less efficient than the phosphorothioate modifications more broadly utilized therein. An end cap might also be constructed of unmodified nucleotides, arranged in a self-complementary foldback structure to provide adequate degradative stability.

An example of a theoretical 25 base pair small end capped single stranded oligonucleotide for use in the therapy of beta thalassemia would have a sequence of:

C*A*A*GGTGAACGTGGATGAAGTT*G*G*T) (SEQ ID: 1)

This correction at base 279, repairs the 1 bp insertion (FS21G) resulting in one of the forms of beta thalassemia (Genbank accession# L48219). Each end would be capped by three phosphorothioate linkages (*) between each of the three end nucleotides to impart the necessary stability against degradation.

Preliminary studies utilizing an end-capped single stranded oligonucleotide of 25 bases in length have demonstrated significant levels of β-globlin gene conversion from normal sequence (β^{a}) to sickle sequence (β^{s}) in blood stem cells microinjected with the oligonucleotide. The end-capped single stranded oligonucleotide is designed to target an A to T transversion in the sixth codon of exon 1 of the β-globin gene locus. An example of the structure of oligonucleotide is as follows:

G*C*A*TCTGACTCCTGAGGAGAAGT*C*T* (SEQ ID: 2)

Where underlined nucleotide represents nucleotide targeted for conversion/transversion and (*) represent end-capped linkages to provide stability to the molecule.

Human blood stem cells (Lin ⁻CD38⁺ phenotype) were microinjected with end-capped single stranded oligonucleotide at a concentration of 2000-5000 copies/fL. The microinjected cells were expanded in vitro and analyzed for conversion of the β-globin sequence by DdeI restriction enzyme digestion of amplified product. In some of these studies, conversion of β-globin from normal sequence to sickle sequence occurred in about 1% of the injected cells. This percentage of converted cells in this target population represents a significant improvement over prior reports of gene conversion attempts employing techniques other than those described here as part of the invention. It is anticipated by the present inventors that the technique defined here may be used to convert a significant percentage of cells in a target population of diseased cells, such as sickle cells, to include cells that have a wild-type genetic make up. As such, the disease state may be corrected for many other genetic diseases.

### EXAMPLE 3

### Demonstration of Gene Conversion/Modification via Single-Stranded End-Capped Oligonucleotide-Mediated Targeted Gene Conversion and Retention of Stem Cell Activity

The present example demonstrates the utility of the present invention for creating a site directed and specific modification into a normal gene sequence.

As an exemplary targeted genetic change, the present example describes the use of single-stranded end-capped oligonucleotide-mediated targeted gene conversion of the β-globin gene locus from normal to sickle sequence (β^{a} to β^{s}) in human hematopoietic stem cells. The targeted nucleotide is an A to T transversion in the sixth codon of exon 1 of the β-globin gene locus. In this example, the technique used to incorporate material into the targeted cell is microinjection. After injection of the desired nucleic acid and/or other accessory molecules, the injected stem cell will have a retention of stem cell activity. This technique may be used, for example, in reconstitution of the human blood system after transplantation of the modified stem cells.

Single-stranded end-capped oligonucleotide DNA molecules designed to convert the normal β-globin sequence (β^{a}) to sickle β-globin sequence (β^{s}) will be delivered (at a concentration of 500-20000 copies per femtoliter) to the immobilized cells via glass needle mediated microinjection. The modified cells will be released by any of a variety of detachment protocols (for example, addition of peptide “cocktail”, mechanical disruption, competition peptides, enzyme action, etc.). Modified cells (e.g. CD34⁺/KDR⁺/CD38⁻LIN⁻) will be injected into NOD/LtSz-Prkdc^{scid} B2m^{tm1Unc}/J mice (NOD/SCID B2m) with or without exogenous cytokines. The NOD/SCID B2m mouse is an immunodeficient mouse that after sublethal irradiation will allow for the engraftment of human hematopoietic stem and progenitor cells.

The B2m^{tm1Unc} targeted mutant strain was generated by a targeted disruption of the B2m gene using the 129-derived E14TG2a ES cell line. The double mutant was generated by backcrossing the B2m^{tm1Unc} mutation 10 generation to the NOD/LtSz-Prkdc^{scid}/J strain. The NOD/SCID B2m model is superior to previous immunodeficient mouse models of human cell engraftment (e.g. the standard NOD/SCID mouse) since there is also an absence of β-2 microglobulin, hemachromatosis, a lack of NK cells, T and B cells, and of complement. Only homozygous mice are used. Genetically modified cells will be injected into sublethally irradiated, or in some cases non-irradiated NOD/SCID B2m mice, with a dose (or range of doses) of treated/corrected cells previously determined to achieve good engraftment in the mice. The bone marrow will be harvested after 6-8 weeks and the expression of human CD45 will be examined in bone marrow mononuclear cells to determine the percent human cells present. Alternatively, the presence of human cells will be assessed by specific detection of human DNA sequences. As well, the human derived blood cells will be analyzed for presence of sickle β-globin sequence. It is anticipated that a detectable percentage (minimum of 1%) of the human blood stem cells and/or their progeny will contain sickle β-globin sequence. Sickle β-globin sequence in human blood stem cell isolated from the mouse bone marrow will demonstrate not only conversion of the β-globin locus in human blood stem cells and their progeny but also retention of stem cell activity in those converted/modified cells. It would be considered accomplished if at least 1% to 10% cells are converted/modified stem cells. This would be considered a significant improvement over past techniques that typically report only a conversion rate of 0.000001 to 0.01% converted stem cells.

### EXAMPLE 4

### Correction of the Sickle Cell Disease Mutation in Human Hematopoietic Stem Cells

The present example demonstrates the utility of the invention for single-stranded end-capped oligonucleotide-mediated targeted gene correction of the sickle cell mutation on the β-globin gene locus (β^{s} to β^{a}) in human hematopoietic stem cells via microinjection, and the retention of stem cell activity in said corrected cells in an animal model of human engraftment. Single-stranded end-capped oligonucleotide DNA molecules designed to correct the sickle cell disease mutation in β-globin will be delivered (at a concentration of 500-20000 copies per femtoliter) to the immobilized sickle cell patient-derived hHSCs via glass needle mediated microinjection. A population of treated cells comprising the corrected cells will be released by one of any variety of detachment protocols (i.e. addition of peptide “cocktail,” mechanical disruption, competition enzyme, etc.). Corrected cells will be pooled (total of 1000-2000 injected cells) and delivered to sublethally irradiated NOD/SCID B2m mice by intravenous (i.v.) injection. FACS and/or PCR analyses will analyze bone marrow collected from the tibia and femurs of engrafted mice for the presence of human derived blood cells. As well, the human derived blood cells will be analyzed for correction or presence of the normal β-globin sequence. It is anticipated that a detectable percentage (minimum of 1%) of the human derived blood stem cells will contain sickle β-globin sequence. Sickle β-globin sequence in human derived blood stem cells and their progeny isolated from the NOD/SCID B2m mouse bone marrow will demonstrate not only conversion of the β-globin locus in human blood stem cells but also retention of stem cell activity in those converted/modified cells. Also, erythroid progeny derived from human stem/progenitor cells will demonstrate conversion of the β-globin mRNA.

### EXAMPLE 5

### Modification of Beta-Globin Locus in Human Hematopoietic Stem Cells

The present example demonstrates the utility of the invention for single-stranded end-capped oligonucleotide-mediated targeted gene modification/replacement of the β-globin gene locus in human hematopoietic stem cells via microinjection. Application of a single-stranded end-capped oligonucleotide strategy to sickle cell gene therapy in a clinical setting requires optimization of the frequency and consistence of gene targeted conversion in human blood stem cells as well as demonstration of β-globin gene conversion and translation of that conversion into functional β-globin protein in the progeny of said modified cells and a resultant phenotypic effect. DNA molecules generated for targeted conversion of β^{s} to β^{a}-globin will be delivered to hematopoietic stem cells (e.g. Lin⁻CD38⁻ phenotype) by glass needle-mediated microinjection. Lin⁻ CD38⁻ cells will be purified from mononuclear cells (MNC) using negative selection with the StemSep® system according to the manufacturer's protocol (Stem Cell Technologies Inc, Vancouver, Canada). The antibody cocktail that removes cells expressing CD2, CD3, CD14, CD16, CD19, CD24, CD36, CD38, CD45RA, CD56, CD66b, or glycophorin A. Lin⁻CD38⁻ cells will be attached to retronectin-coated dishes by incubation of the cells on a plate for 45 minutes at 37° C. DNA molecules will be introduced into the attached cells by glass-needle mediated microinjection.

Cells will be injected with borosilicate glass needles (outer tip diameter 0.17-0.30 microns) under an Olympus IX70 inverted microscope with the electronically interfaced Eppendorf Micromanipulator (Model 5171) and Transjector (Model 5246). The success of injection of the blood stem cells as determined by % viability (number of fluorescent cells/number of successfully injected cells×100) typically ranges from 70-90%. Injected cells will be detached by addition of peptides (fibronectin CS-1 fragment, VLA-4 inhibitor peptide, and RGDS peptide at 5 mg/ml each) as described in Davis et al. (2000), and transferred either as total injected cell samples to wells of 48 well plates or individually injected cells using a Quixell™ Automated Cell Selection and Transfer Unit (Stoelting, Wood Dale, Ill.) to wells of 96-well plates. The former will be grown for 4-6 weeks in media containing EPO (50U/ml), IL-3 and GM-CSF as described in Malik et al. (1998) to promote expansion and differentiation of blood stem cells to erythroid lineages. The latter (the individually transferred cells) will be cultured in IMDM media supplemented with bovine serum albumin, insulin, transferring, low density lipoprotein, 50 mM HEPES (pH 7.4), Stem Cell Factor (SCF, 100 ng/ml), Flt-3 ligand (100 ng/ml), IL-3, IL-6, G-CSF (20 ng/ml) and β-NGF (5 ng/ml) which promotes hematopoietic cell expansion and differentiation along multiple lineages. Individually transferred cells are grown in this highly enriched cytokine media to insure maximal survivability of the stem cells in a clonal environment.

The differentiated and expanded cells will be assessed for gene targeted conversion of the β-globin locus from β^{a} to β^{s} sequence at the DNA, RNA and protein level. Analysis of the β-globin mRNA will be performed by RT-PCR and Dde1 digestion of β-globin sequences. Loss of a Dde1 digestion site is indicative of the conversion from β^{a} to β^{s} globin sequence following injection of DNA molecules. These studies are essential to demonstration that the gene conversion observed at the DNA level in blood stem cells and progeny is translated to altered RNA sequence and ultimately protein. As such, cell samples demonstrating conversion at the DNA level in the β-globin locus will be further analyzed for presence of conversion in β-globin mRNA and β-globin protein.

Presence of sickle and normal β-globin protein in injected cell samples will be analyzed by a variety of techniques. Firstly, β-globin protein isolated from erythroid expanded cells will be analyzed for the presence of β^{s} globin by HPLC. High pressure liquid chromatography (HPLC) is a highly sensitive, rapid, and reproducible technique capable of differentiating among many abnormal hemoglobins. Briefly, 5×10⁵ cells will be isolated and analyzed. Secondly, β-globin protein will be analyzed for the presence of β^{s} globin by sickle and normal globin specific antibodies in immunoassays. An alternate method for the detection of sickle globin in blood samples is a traditional electrophoresis method including alkaline pH on cellulose acetate followed by further examination of abnormal samples by acid electrophoresis on citrate agar. These techniques provide reliable detection of hemoglobins (Hb) S, C, and A even in the presence of large amounts of Hb F. These methods have been modified and are now commercially available as pre-cast gels (Helena, Beaumont, Tex.).

### EXAMPLE 6

### Use of Single-Stranded End-Capped Oligonucleotide Technology for Generating Primary Cells or Cell Lines with Defined Mutations for Use in the Field of Functional Genomics

Deciphering the human genetic code will eventually reveal the individual genes responsible for numerous diseases. However, identifying the gene sequence is only the beginning of the challenge. Once a gene sequence is identified, researchers must determine the physiological function of that particular gene. Increasingly, it has become evident that a given gene may vary by only a collection of single base changes from individual to individual (referred to as single nucleotide polymorphisms, or SNPs). These variances may be key for understanding why not all individuals exhibit the same disease symptoms, and why some patients respond better to some treatments than others. Cataloguing these SNP variances in the form of a library holds the promise of ‘tailoring’ treatments to suit each individual; a library of gene-modified cells could be employed for targeting therapeutics to individuals with specific mutations, ushering in a new age of personalized medicine.

To create mutant cell lines, two specific techniques can be used: the knock-out and the SNP (single nucleic acid polymorphism). The former interrupts the target gene's expression by inserting a stop codon in the gene, prohibiting expression of the full-length protein and instead producing a truncated, inactive protein. This essentially results in turning off the gene. The latter alters the sequence of the gene by introducing a specific mutation into the normal gene sequence. The gene continues to express itself but the resulting proteins are altered at one or more positions in the sequence of amino acids comprising the protein. Both types of genetic alteration allow the investigator to observe resulting changes and identify gene function. The present invention could be employed to produce primary cells or cell lines containing defined alterations in target DNA sequences. Such modified cells could be used to assess gene function in the case where function is unknown or suspected (“target gene validation”) and/or where the effect of specific mutations on gene function.

This example contemplates the use of single-stranded end-capped oligonucleotides to generate a cell line containing a specific genetic modification (e.g. mutating a wt sequence or converting a mutant sequence to the wt).

### EXAMPLE 7

### Use of Single-stranded end-capped oligonucleotide Technology to Create Transgenic Animals

The present example illustrates the utility of the invention in the field of transgenics. A method of rapidly and efficiently changing specific sequences of DNA in cells or animals and observing the results is essential for functional studies. Similarly, for transgenics, embryonic stem cells modified by the method of single-stranded end-capped oligonucleotides could be used to produce animals containing modified sequences in specific genes. These animals could be used as models of human disease. As well, the growth, development and survival of the animals would be monitored to study the effects of the specific gene alterations.

Transgenic mice can be generated by either incorporating genetic material directly into fertilized mouse eggs by injection, or by incorporating genetic material into mouse ES cell lines by microinjection or electroporation. Transgenic mouse models of SCD have been developed to elucidate the pathophysiology of the disease and to test potential therapeutic approaches (Blouin, M. J., et al. (2000), Nature Medicine, 6:177-182; Ryan, T. M., et al. (1997), Science, 278:873-876; Paszty, C., et al. (1997), Science, 278:876878). However, the current methods used to develop viable transgenic mice, containing a defined mutation of a specific gene or the knock-out of a gene, requires an enormous amount of time and effort, and is relatively costly. Only approximately 1-5% of all attempts to insert human DNA into fertilized mouse eggs is successful, and even if the gene transfer is successful, implantation of the embryo is not guaranteed, and further more, the introduced genes sometimes fail to function in the animal.

The application of single-stranded end-capped oligonucleotide technology to the generation of transgenic mice will greatly increase the efficiency and speed of strain production, efficacy of the genetic modification, and reduce the cost, since the need for crossing and back-crossing various strains will not be necessary, in the case where both alleles are modified.

To create mutant mouse ES cell lines, two specific techniques can be used: the knock-out and the SNP (single nucleic acid polymorphism). The former interrupts the target gene's expression by inserting a stop codon in the gene, prohibiting expression of the full-length protein and instead producing a truncated, inactive protein. This essentially results in turning off the gene. The latter alters the sequence of the gene by introducing a specific mutation into the normal gene sequence. The gene continues to express itself but the resulting proteins are altered at one or more positions in the sequence of amino acids comprising the protein. Both types of genetic alterations can be performed using Single-stranded end-capped oligonucleotide technology and allow the investigator to observe resulting changes and identify gene function, a process referred to as “target gene validation”. Demonstrating an improvement over currently utilized methods of producing genetically modified animals.

This example contemplates utilizing single-stranded end-capped oligonucleotides to generate mouse models of various hemoglobinopathies such as, sickle cell trait, sickle cell disease, and forms of thalassemia. Sickle cell trait would be readily generated by introducing a mutant sequence into only one allele of the hemoglobin β gene. Conversion of both alleles by single-stranded end-capped oligonucleotides, or by crossing resultant heterozygotes, would produce the genotype of sickle cell disease.

In one form of the disease thalassemia (Cotran, R. S., et al. (1999), Pathologic Basis of Disease, (6th Ed., W. B. Saunders Company, Philadelphia, Pa.), a deletion occurs in the hemoglobin β gene resulting in a loss of production of this form of hemoglobin. This particular gene defect would be generated in a mouse model using single-stranded end-capped oligonucleotides by converting wild type mouse sequences to generate a stop codon, thereby, inhibiting production of the protein and mimicking conditions of the disease.

### EXAMPLE 8

### Gene Therapy Method for Correcting an Inherited or Acquired Genetic Disease

The present example demonstrates the utility of the invention for providing an effective mechanism for genetically modifying undifferentiated cell types, such as human blood stem/progenitor cells. In this manner, the present application also demonstrates the utility of the present invention for a method to provide gene therapy using cells that are modified at the β-globin locus for treatment and/or cure of diseases including but not limited to Sickle Cell Disease (SCD).

Treatment of sickle cell disease by gene therapy will best be accomplished by repair of the genetic defect in the β-globin gene. A single base pair mutation in exon 1 of the β-globin gene leads to the synthesis of an abnormal protein, “sickling” of red blood cells, and ultimately, disease. Gene repair is ideal for correction of small genetic changes, as gene repair technologies employ natural cellular processes in the correction of the disease-causing mutation within the human genome. Repair of the mutation within the β-globin gene (gene repair strategies), as opposed to introduction and/or integration of an exogenous normal β-globin gene (gene compensation strategies) will insure appropriate β-globin expression in red blood cells and disease treatment.

Human hematopoietic stem cells (hHSCs) (e.g. CD38⁻/lin⁻/CD34⁺/KDR⁺ cells) would be isolated from the bone marrow of a sickle cell anemia patient. Approximately 1-100×10³ highly enriched hHSCs will be temporarily immobilized by the methods described in the parent application, U.S. Ser. No. 09/336,655. Single-stranded end-capped oligonucleotides DNA molecules will be delivered to the immobilized hHSCs. The delivery method of microinjection allows for defined and accurate delivery of specific quantities of the single-stranded end-capped oligonucleotide DNA molecules to the nucleus of the hHSCs. For example, 2500 copies/fl will be delivered to each hHSC with an approximate volume of 0.5-2 fL being introduced into each hHSC. Single-stranded end-capped oligonucleotide DNA molecules will be delivered to the maximal number of cells immobilized. Following injection the cells will be detached by methods described. An aliquot of the treated hHSCs will be retained in culture for analysis of gene replacement in the β-globin DNA, RNA, and protein. The gene-modified blood stem cells would be returned to the patient (either with or without ex vivo stem cell expansion) where the repaired blood stem cells could re-establish the blood system with red blood cells containing normal β-globin protein. It is estimated that beneficial effects will be observed in sickle cell disease patients if approximately 10% of the circulating red blood cells contain healthy, normal β-globin. Therefore, repair of 10% of the blood stem cell population would allow for a continual and sufficient supply of corrected, healthy red blood cells. Importantly, healthy red blood cells have a significantly longer half-life in circulation as compared to sickle red blood cells (120 days for healthy red blood cells versus 20 days for sickle red blood cells) suggesting a selective advantage for healthy red blood cells may exist. This selective advantage suggests that repair of only 2-4% of the blood stem cell pool may provide significant therapeutic benefit to patients.

The single base pair mutation responsible for SCD occurs on both copies or alleles of the β-globin gene in all nucleated cells within the patient. The sickle β-globin is designated by β^{s} β^{s}. Repair or correction of one of the copies or one of the alleles (i.e. conversion from β^{s} β^{s} to β^{s} β^{a}) is sufficient for production of adequate levels of correct β-globin protein and healthy red blood cells in a patient. As such, the treated/corrected cell population will comprise cells having the genotype β^{s} β^{s} (as not 100% of the cells in the population will be modified), β^{s} β^{a}, and β^{a} β^{a} (correction/repair of both alleles may be possible).

### EXAMPLE 9

### Chemoprotection of Host Blood Cells During Exposure to Chemotherapeutic Agents

The present example demonstrates the utility of the present invention for using the herein described methods for creating specific, targeted mutations in a gene without reducing the function of that gene in the animal, while imparting a resistance to the gene that enables it to resist the challenge of typical agents used in the chemotherapy of patients, such as in the treatment of various cancers.

The present example outlines one embodiment whereby this approach may be used to impart resistance to those chemotherapeutic agents classified as antifolate drugs, such as methotrexate and trimetrexate, to a gene in a cell, using gene repair techniques. Other techniques, such as gene compensation, may be used to create cell resistance to chemothereapeutic agents as well.

According to this aspect of the invention, cells may be modified so as to be resistant to chemotherapy, protection/resistance from specific chemicals (chemotherapeutics), radiation, specific chemicals, and infectious agents such as HIV.

Antifolate drugs such as methotrexate (MTX) exert their antiproliferative effect through competitive inhibition of the cellular enzyme dihydrofolate reductase (DHFR), which is essential for de novo synthesis of thymidylate and purine nucleotides. MTX is the most frequently used drug in this class for cancer treatment, and is also used in the treatment of nonmalignant conditions. The clinical usefulness of MTX is limited both by the emergence of drug resistant tumor cells and by toxicity to normal host tissues, especially to bone marrow. Trimetrexate (TMTX) is a newer antifolate that offers attractive properties for clinical use. Because TMTX can passively enter cells that have become resistant to MTX through alterations in the folate active transport mechanism¹, TMTX is active against certain MTX-resistant tumors (Kheradpour, A., et al. (1998), Cancer Invest, 13:36). In clinical trials, TMTX has shown activity in some advance pediatric and adult tumors, but its use was often limited by severe myelosuppression (Witte, R. S., et al. (1994), Cancer, 73:688; Lacerda, J. F., et al. (1995), Blood, 85:2675).

One approach for increasing the therapeutic index of TMTX would be to introduce antifolate-resistant DHFR genes into bone marrow cells. A mutant DHFR (L22Y) gene has been identified to provide a high level of TMTX resistance in both murine fibroblasts and hematopoietic progenitor cells (Fry, D. W., et al. (1988), Cancer Res, 48:6986). By gene repair, using small single stranded end-capped oligonucleotides that are homologous to the endogenous DHFR gene except at codon 22, a mutation may be introduced in the endogenous DHFR gene and make it highly resistant to TMTX and other antifolate drugs.

### EXAMPLE 10

### Advantage of Microinjection in the Co-Introduction of Accessory Molecules with Single-stranded end-capped oligonucleotide Molecules for Genetic Modification

The present example demonstrates the utility of the invention for the co-introduction of single-stranded end-capped oligonucleotides and accessory molecules, including but not limited to proteins, for increasing the efficacy of gene repair mediated by single-stranded end-capped oligonucleotide molecules.

Accessory molecules include, but are not limited to those included in the following groups. First, a protein whose function is to alter the conformation of DNA to recombination forms either as monomers, polymers, or protein complexes. Secondly, proteins whose natural enzymatic functions are involved in the proofreading, excision, or repair of the genomic DNA. Third, any and all proteins involved in the regulation therein of any other component of such recombination or repair pathways involved in the expression, integration, recombination, or functioning of the pathways necessary for the effect of the delivered therapeutic nucleic acid molecules.

The role of native accessory proteins in the mediation of gene repair effects has been demonstrated in Cole-Strauss et al (1999), in which gene repair effects on point and frame shift mutations for the original chimeric molecule are shown to be affected by the presence of the hMSH2 protein. MSH2 is a member of the mismatch repair pathway, and acts in the initialization of the repair process. Although Gamper et al (2000) demonstrated the repair using the single stranded end-capped oligonucleotides was possible even in the absence of functional MSH2, this does not eliminate a possible positive influence of the protein upon this baseline of activity if co-introduced. More importantly, the different observations of Cole-Strauss et al (1999) and Gamper et al (2000) clearly show that the precise mechanism of repair is highly complicated and non-fully characterized at this point. Hence, which added accessory molecule will yield the highest increase to the repair effects of the single stranded end-capped oligonucleotides will have to be determined experimentally from a large number of potential candidate molecules.

The co-introduced proteins may include, but are not limited to, recombinogenic molecules intended to stimulate homologous pairing and recombination such as RecA, Rec2/Rad51L and other members of the Rad51 group of proteins including, but not limited to, human and yeast forms of the proteins. Other co-introduced proteins that will hypothetically facilitate correction frequencies will be those specifically used to increase the efficacy of the mismatch repair pathway, specifically, but not limited to, hMSH2, hMSH3, or MutS.

Particular co-introduced molecules, such as, but not limited to proteins such as Rad 51 operate at specific concentration ratios with the introduced therapeutic molecule. A ratio with too little accessory molecule will not be able to function, due to side reactions, such as self-association of the accessory molecule. Other methodologies cannot control this ratio once exposed to the target cells, as the single-stranded end-capped oligonucleotide and accessory molecule will be separated and will not reach the nucleus of the cell in the same ratios as originally composed. Co-introduction of the single-stranded end-capped oligonucleotide and the accessory molecules via microinjection will provide a controlled ratio to be directly introduced into the nucleus of the hHSC.

Similarly, other methodologies allow for the sequestration and separation of therapeutic molecules and accessory molecules amongst various compartments within the cell, in which modification, cleavage and inactivation of both molecules can occur. By directly injecting into the nucleus both the single-stranded end-capped oligonucleotide and the accessory molecule, the possibility of cleavage, segregation, or inactivation of one or both components is avoided.

### EXAMPLE 11

### Co-introduction of Single-Stranded End-Capped Oligonucleotide Molecules and Accessory Oligonucleotides into Cells for Genetic Modification into Human Hematopoietic Stem Cells

This example describes the utility of the invention for the co-delivery of single-stranded end-capped oligonucleotide molecules and accessory oligonucleotides into a cell for the purpose of increasing the efficiency of gene repair. Levels of correction can be increased by the co-introduction of specific nucleic acid oligonucleotides designed to affect the inherent mechanisms of the homologous recombination and mismatch repair pathways. These two DNA repair pathways have been generally accepted as the primary mechanisms by which therapeutic gene repair takes place. The co-delivery of these molecules into hHSCs can be accomplished using techniques as described in previous examples here including, but not limited to, microinjection.

By means of an example, these molecules include, but are not limited to, antisense oligonucleotides to suppress the genes of one DNA repair pathway for the purpose of up-regulating a competing repair pathway that mediates the therapeutic recombination of the introduced single-stranded end-capped oligonucleotide molecules. Suppression using anti-sense oligonucleotides has previously been shown by the suppression of components of the Rad 52 epistasis group, notably HsRAD51 (Xia et al, Molecular and Cellular Biology 17(12):7151-8,1997 December). Inactivation of primary molecules in the homologous recombination pathway have been shown to up-regulate mismatch repair pathway components. Evans E, et al, (Molecular Cell, 5(5) 789-99, 2000 May) showed that the mismatch repair component, Msh2p, was up-regulated in rad52 deficient yeast strains. Therefore, by means of an example, therapeutic single-stranded end-capped oligonucleotide molecules will be co-introduced into hHSCs with anti-sense oligonucleotides to Rad 51, an active recombinase of the Rad52 group or other components of it's repair pathway. The effected suppression of the homologous recombination pathway will allow for an up-regulation of components in the mismatch repair pathway, yielding a higher efficacy of repair mediated by the single-stranded end-capped oligonucleotides.

By means of another non-exclusive example, co-introduction of single-stranded end-capped oligonucleotide molecules with oligonucleotides designed to provide additional substrates for stimulation of elements of either recombination mechanism might likewise increase the efficacy of gene repair. These molecules are herein defined as “decoy molecules”. Additional substrate can be provided by nonsense oligonucleotides specifically designed with specific lesions and mis-pairing of nucleotides in order to stimulate the up-regulation of proteins associated with repair pathways, such as the mismatch repair pathway. These up-regulated pathways, having increased the amount of their active components after stimulation, are then the mediating pathways for the therapeutic recombination of the single-stranded end-capped oligonucleotide molecule with the sequence of interest in the genome. In this manner, single-stranded end-capped oligonucleotide molecules will be co-introduced with these stimulatory oligonucleotides by methods described here earlier including, but not exclusive to, microinjection. The effect will be an increased level of cellular molecules associated with the repair pathway, thereby mediating a higher efficacy of repair mediated by the single-stranded end-capped oligonucleotide molecule.

Small oligonucleotides have been shown above to be capable of gene repair of the β-globin gene in CD34+ enriched cells. Single-stranded end-capped oligonucleotide molecules and accessory proteins will be introduced into human hematopoietic stem cells (e.g. CD34+38−, CD38−/Lin−, CD34+/KDR+/CD38−/Lin−, CD34+/KDR+/CD38−/CD33/Lin−) in order to facilitate a point mutation correction of the β-globin gene for the correction in progeny cells of sickle cell disease at a clinically applicable frequency. These single-stranded end-capped oligonucleotide molecules will be introduced in concentrations ranging between 500 and 20,000 copies per femtoliter.

The mechanism of co-introduction includes, but is not limited to, glass-needle mediated nuclear microinjection. Other delivery techniques include electroporation, liposomal transfection, laser mediated introduction (such as, but not limited to the laser scissor and optical tweezer methods), and particle bombardment.

### EXAMPLE 12

### Single-Stranded End-Capped Oligonucleotide-Mediated Targeted Gene Modification of the β-Globin Locus (Normal to Mutant (Sickle) Sequence)

Human blood stem cells (hHSCs) were isolated from the umbilical cord blood of a healthy, normal donor. The normal hHSCs were attached to matrix-coated surfaces and microinjected with the gene repair molecules. A range of 5-90% of the attached hHSCs were injected with the repair molecules. Following injection, the cells were detached, removed from the matrix-coated dishes, and cultured in vitro using conditions designed to promote expansion and differentiation. Genomic DNA or cell lysates were obtained from the expanded cell cultures and used for molecular analysis by the polymerase chain reaction (PCR) amplification of β-globin sequences.

a) Allele-specific PCR amplification analysis utilizes the ability of specific primers (SC9A and SC9S) to distinguish between the normal and sickle β-globin gene sequences. Primer SC9A (in combination with the reverse primer SC4 specifically recognizes the normal β-globin sequence and only allows for the PCR amplification in samples containing normal β-globin sequence. Primer SC9S exclusively recognizes sickle β-globin sequence and only allows for PCR amplification in samples containing sickle β-globin sequence. Cell samples that contain both normal and sickle β-globin sequence generate PCR amplified product with both SC9A/SC4 and SC9S/SC4 primer sets.

b) Restriction enzyme digestion analysis exploits the ability of restriction enzymes to recognize and cut specific gene sequences. Normal β-globin gene contains a sequence that is recognized by the restriction enzyme Dde1. When normal β-globin is converted to sickle β-globin, the specific sequence change occurs at the region coinciding with the Dde1 enzyme recognition site; as a result, sickle β-globin is no longer recognized by the Dde1 enzyme at that one particular site. Hence, Dde1 can be used to distinguish between normal and sickle β-globin. Isolated genomic DNA or total cellular lysates from expanded hHSCs microinjected with gene repair molecules, were subjected to PCR amplification using the primer set SC3/SC4 (amplification of both normal and sickle β-globin sequences). Amplified product was then digested with Dde1. Samples containing normal β-globin sequence generate 3 unique digestion products whereas samples containing sickle β-globin sequence generate 2 unique digestion products. Samples containing both normal and sickle sequences generate a mixture of the normal and sickle digestion patterns.

c) Sequence analysis of PCR amplified β-globin sequences involves TA-cloning and sequence analysis. This technique allows for estimation of the frequency of gene conversion in hHSCs microinjected with gene repair molecules.

Conversion of the β-globin gene from normal to sickle sequence was demonstrated by allele-specific PCR analysis and confirmed by restriction enzyme digestion analysis and sequence analysis.

### EXAMPLE 13

### Single-Stranded End-Capped Oligonucleotides in Treatment of AIDS

This example describes the utility of the invention for single-stranded end-capped oligonucleotide-mediated therapy of HIV infection by treatment of hHSCs. The HIV virion binds to the CD4 receptor on cell surfaces for primary binding, but requires a secondary co-receptor for internalization into the attacked T-cell. By way of an example of such a co-receptor is the cytokine receptor CCR5. Polymorphisms in this receptor have been shown to alter the efficacy of both infection and anti-retroviral therapeutics to the infection (Efremov, R, et al. (1999), European Journal of Biochemistry, 263:746).

As described in earlier examples, hHSCs will be isolated from either the patient or a suitable donor and purified to a stem cell population, for example hHSCs with markers of CD34⁺/CD38⁻lin⁻. As described in earlier examples herein, single-stranded end-capped oligonucleotide molecules will then be introduced into hHSCs. This introduction into the hHSCs may be accomplished via any of the techniques previously described herein, including, but not limited to, microinjection following attachment of the hHSCs via the technique likewise described earlier.

These single-stranded end-capped oligonucleotide molecules will be encoded with sequence changes designed to introduce changes similar to the polymorphisms seen in HIV resistant examples of co-receptors. By means of such an example, such a change will be in hydrophobicity motifs in the first extra cellular loop region of the CCR5 coreceptor. Such an introduction of a mutation in the extracellular domain of the CCR5 cell surface receptor mediated by single-stranded end-capped oligonucleotide molecules to a sequence matching a polymorphism previously shown to inhibit infection of T-cells, will result in an inability of the HIV virion to infect the hHSC via that co-receptor, giving it a measurable degree of resistance.

Treated cells will then be detached and transplanted into the patient or may first be treated in culture with techniques, as described earlier, to either expand hHSCs or to select for a particular lineage. By means of example, the treated hHSCs could then be treated with the cytokines IL-1 and IL-6 in order to select for a lymphoid stem cell lineage, which would then produce HIV resistant B cells and T cells. Cells would be expanded as previously described herein to sufficient numbers for re-introduction into the patient and re-establishment of the patient's hematopoietic immune system.

### EXAMPLE 14

### Utilization of Therapeutic Single-Stranded End-Capped Oligonucleotide Molecules in Non-Hematopoietic Stem Cells

This example describes the utility of the invention for single-stranded end-capped oligonucleotide-mediated therapy in non-hematopoietic stem cells. By means of an example, the therapy of hepatic stem cells will be described, though the potential applications using these molecules in non-hematopoietic stem cells includes, but is not limited to, these examples in hepatic stem cells given.

Hepatic stem cells, or Oval Cells, and their precursors can be found in bone marrow, and can be identified as having hematopoietic-like identifiers, such as CD34⁺ and c-kit (Petersen, B. E., et al. Science, 284:1168-70, May 14, 1999) in association with non-hematopoietic cytokeratin markers CAM 5.2 and CK 8 and 18 (Lemmer, E. R., et al. (1998), Journal of Hepatology, September 29(3):450-4). Thusly, hepatic stem cells can by isolated from bone marrow and purified using methods as described previously herein.

Ornithine Transcarbamylase Deficiency (OTC) is an X-linked disorder, whose causality varies among several point mutations and deletions leading to hyperammonemia, pronounced orotic aciduria and an abnormal phenotype characterized by growth retardation. Correction of this disorder in OTC deficient mice has been shown via microinjection of a construction of rat OTC cDNA into the germ line prior to development (Cavard C, et al, Nucleic Acids Research, 1988, Mar 25;16(5):2099-110). While this shows that correction is possible, this approach does not address gene therapy techniques suitable for treatment of an adult patient. However, gene therapy approaches using single-stranded end-capped oligonucleotide molecules and the techniques previously described herein offer opportunity for the genetic treatment of oval cells and their precursor cells for the treatment of OTC among other disorders.

By means of an example, oval stem cells will be isolated from the bone marrow of patients, by selection for the markers listed above using techniques described previously herein. Single-stranded end-capped oligonucleotide molecules will then be introduced into these purified oval cell precursors for the purpose of genetic alteration of a hepatic dysfunction such as OTC.

The single-stranded end-capped oligonucleotide molecule introduced may mediate varied effects, such as the alteration of CpG dinucleotide of codon 141 from the altered CAA back to the functional CGA sequence. This alteration has been shown to be the case in approximately 10% of all the OTC cases, so is an example, but by no means is the only manner in which single-stranded end-capped oligonucleotide molecules may mediate a therapeutic effect. The precise sequence and effect mediated by the single-stranded end-capped oligonucleotides for therapy of oval precursor cells will have to be varied according to the precise genetic malady of the patient. As another example would be single-stranded end-capped oligonucleotide mediated repair of the C to T transition in codon 109 (Maddalena. A., et al. (1988), Journal of Clinical Investigation, October; 82(4): 1353-8) or correction of the Leu148Phe substitution as noted by Matsuura K. S., et al. (1997), American Journal of Medical Genetics, March 17;69(2):177-81.

After introduction of the single-stranded end-capped oligonucleotide molecules, the cells will be detached and cultured using methods either similar to those described previously herein or specific to expansion of oval stem cells before being re-introduced into the patient. Once re-introduced into the patient, the defining markers of the cell will theoretically target the cells from general circulation into residency in the liver, establishing a genetically corrected population of cells for corrected hepatic function.

### EXAMPLE 15

### Use of Single-Stranded End-Capped Oligonucleotide Technology for Generating Primary Cells or Cell Lines with Defined Mutations for use in the field of Functional Genomics

With the sequencing of the human genome almost completed, the combination of DNA array technology, high throughput screening systems, and sophisticated bioinformatics, the discovery of complex genetic components of disease will proceed rapidly. Common polymorphisms, including SNPs, in drug targets have been linked to altered drug sensitivity. For example, polymorphisms in the angiotensin converting enzyme (ACE) affects its sensitivity to ACE inhibitors (Henrion, D., et al. (1998), Journal of Vascular Research, 35:356). Genetic polymorphisms underlying disease pathogenesis can also be determinants of drug efficacy. The risk of adverse drug effects has also been linked to genetic polymorphisms (e.g. dopamine D3 receptor and the risk of drug-induced tardive dyskinesia (Steen, V. M., et al. (1997), Molecular Psychiatry, 2:139). While the list of SNPs and polymorphisms grows, it is not always clear which DNA sequence variations contribute to, or are responsible for the effect (or lack thereof) of a particular drug on a target population. This example contemplates the generation of cells or cell lines with defined mutations for use in the field of pharmacogenomics. Specific mutations can be generated in a receptor of interest expressed by a target cell type (e.g. endothelial cells expressing ACE). The binding of the drug, or potentially inhibition of drug binding to the receptor by a drug antagonist, can be measured in cells containing various SNPs or combinations of SNPs created by the single-stranded end-capped oligonucleotide method. Direct comparisons can be made on the various cell types regarding efficiency of binding and ultimately the effect of the drug. This could be performed in cultures or in single cell assays where high-throughput screening is preferred.

### EXAMPLE 16

### Use of Single-Stranded End-Capped Oligonucleotide Technology for Functional Genomics and Metanomics in Plant Cells

This example describes the utility of the invention for single-stranded end-capped oligonucleotide-mediated therapy in the study of functional genomics and metanomics in plant cells.

The original chimeric molecule was used previously to alter genes in plants. Beetham et al. (1999) used the chimera to create a mutation in the ALS gene of tobacco cells, detectable in the presence of herbicide. Zhu et al (1996) used the chimera to correct a mutation in a fusion gene using a GFP readout system and subsequent inheritance of the correction through demonstrated Mendelian segregation. More recently, Gamper et al (2000) demonstrated the use of single stranded end-capped oligonucleotides in directed gene repair of kanamycin resistance in plant cell-free extracts from Canola and Musa. These results showed a comparable increase in repair activity to those seen in the mammalian cell-free extracts.

The single stranded end-capped oligonucleotides of the present invention can be used to effect normal to mutant and knockout changes in any of a variety of plant cells to determine gene/protein function within a given plant species. Likewise these molecules can be used to determine the effect of a particular protein on a particular pathway in the field currently defined as metanomics. Understandings of these basic gene functions in plants will allow deeper understanding of the mechanisms and pathways of plants, including those responsible for the resistance of certain plant species and breeds to insects, pests, and other pathogens which damage crop species. These plants include, but are not exclusive to, maize (Zea mays), tobacco (Nicotiana tabacum), rice, and banana (Musa acuminata). These are treated in conditions under which the plant cells are treated to remove their cell walls and/or make the cell wall more susceptible to introduction of the single stranded end-capped oligonucleotide and/or accessory molecules to facilitate more efficient gene conversion while the cells are attached and detached to a substrate in a manner analogous to that described earlier in the parent application, U.S. Ser. No. 09/336,655. Such modifications to the parent application's method may include the modification of the substrate to include agar and/or plant lectin binding carbohydrates and their derivatives. The manner of introduction of the single stranded end-capped oligonucleotides includes, but is not limited to, glass needle mediated microinjection. Other methods include, but are not limited to, electroporation, liposomal transfection, laser mediated introduction (such as, but not limited to the laser scissor and optical tweezer methods), and particle bombardment.

### EXAMPLE 17

### Genetically Engineered Plants with the Novel Oligonucleotides

This example describes the utility of the invention for the genetic alteration of plant cells for the purpose of the generation of plants capable of pest and/or pesticide resistance and the capability of the production of plant-derived therapeutics.

Genetic alteration of crop species has become increasingly common place, particularly to introduce genes into these species to impart resistance to crop pests or pathogens. For example, the aromatic rice, Orayza sativa L., has been transformed with the Bacillus thuringiensis (Bt) Berliner cryl1Ab toxin gene under control of the maize phosphoenolpyruvate carboxylase promoter for resistance against various lepidopterous lice pests (Alinia, F., 2000). The use of Bt toxin against pests has become widespread, particularly in rice species, and serves as an example of the effectiveness of recombinant technologies to allow for non-chemical alternatives to conventional pesticides. Another recent example is the introduction of various resistance genes into different raspberry (Rubus idaeus L.) genotypes to impart resistance against the large raspberry aphid, Amphorophora idaei Borner, (Jones, 2000).

In a similar manner, it is proposed the current end capped single stranded oligonucleotides can be used to introduce suitable genetic changes into current plant crop species as to impart resistance against various pests and pathogens. These pests and pathogens include, but are not limited to, insects and other animals, weeds, and inherent microbial and vial pathogens that as such damage, impair, or reduce the crop value or viability of the species. By means of a non-exclusive example, this technology could be used to either introduce, or change the antigenic profile of the Bt toxin introduced or to be introduced into target rice species.

These plants include, but are not exclusive to, maize (Zea mays), tobacco (Nicotiana tabacum), rice, and banana (Musa acuminata). These are treated in conditions under which the plant cells are treated to remove their cell walls and/or make the cell wall more susceptible to introduction of the single stranded end-capped oligonucleotide and/or accessory molecules to facilitate more efficient gene conversion while the cells are attached and detached to a substrate in a manner analogous to that described earlier in the parent application, U.S. Ser. No. 09/336,655. Such modifications to the parent application's method may include the modification of the substrate to include agar and/or plant lectin binding carbohydrates and their derivatives. The manner of introduction of the single stranded end-capped oligonucleotides includes, but is not limited to, glass needle mediated microinjection. Other methods include, but are not limited to, electroporation, liposomal transfection, laser mediated introduction (such as, but not limited to the laser scissor and optical tweezer methods), and particle bombardment.

### EXAMPLE 18

### Microinjection into Plant Cells

The present example demonstrates the advantage of microinjection for the delivery of the single stranded end-capped oligonucleotides into plants cells over current methodologies.

Currently, most genetic manipulations into plant cells are achieved by the introduction of genetic materials via plant bacilli, such as Agrobacterium tumefaciens, as shown in Zhao, et al.(2000). While the bacterium is efficient at the transfection of sustained genetic material into plants, it is unable to do so with either 1) a controlled copy number, or 2) while co-introducing those accessory molecules we believe will be essential for the control of efficient gene targeting techniques, for example via the mismatch repair or homologous recombination pathways. Microinjection directly into plant cells offers a distinct advantage over current methodologies by allowing distinct quantities of intact single stranded end-capped oligonucleotides to be directly introduced into the nucleus of target cells, while allow for the direct co-introduction of accessory molecules at precisely controlled ratios. Microinjection also offers a methodology without the possible side effects of biological transfection, such as the induction of stem-tumors in transfected crops, (Mistrik et al, 2000).

The attachment of these cells can be via agar and/or plant lectin binding carbohydrates and their derivatives. Subsequent detachment, post-treatment, can be achieved via competition by free carbohydrates or chelation co-factors.

### EXAMPLE 19

### Single-Stranded End-Capped Oligonucleotide Constructs

The present example demonstrates the breadth of molecular constructs that may be used in the practice of the invention. As used here, the term “end-capped” is intended to include the addition of nucleotides to the end of a piece of single-stranded nucleic acid, or the chemical modification of both ends of a single-stranded nucleic acid. By way of example, such chemical modifications include a backbone of methylphosphonate, phosphoramididate, morpholino peptide linkages, or containing different 2′-halo, 2′-alkyl, or 2′-alkoxylalklyl sugars. Gamper et al (2000) demonstrated that other modifications can impart such protection, such as their use of 2′-O-methyl ribonucleotides, though such protection was determined to be less efficient than the phosphorothioate modifications more broadly utilized therein. By way of another example, the manner in which an end cap might also be constructed could be of unmodified nucleotides, arranged in a self-complementary foldback structure to provide adequate degradative stability.

### Bibliography

The following references are specifically incorporated herein in their entirety, by reference, for the purposes described herein, and are intended to supplement the present disclosure relative technical material known in the relevant art.

1. Alinia F., Ghareyazie, B., Rubia, L., Bennett, J., Cohen, M B., “Effect of plant age, larval age, and fertilizer treatment on resistance of a cryl1Ab-transformed aromatic rice to lepdopterous stem borers and foliage feeders.” Journal of Economic Entomology. 93(2)484-493. (2000)

2. Bandyopadhyay, P., Ma, X., Linehan-Stieers, C., Kren, B. T., Steer, C. J. “Nucleotide Exchange in Genomic DNA of Rat Hepatocytes Using RNA/DNA Oligonucleotides. Targeted Delivery of Liposomes and polyethyleneimine to the Asialoglycoprotein Receptor.” J. of Biological Chemistry, 274:10163. (1999)

3. Beetham P. R., Kipp, P. B., Sawyky, X. L., Antzen, C., May, G. D., (1999) Proc. Natl Acad. Sci. USA, 96:8774-8778. (1999)

4. Birikh, K. R., Plutalov, 0. V., Schwartz, E. I., Devi, P. S., and Berlin, Y. A. “A Modified Approach to Identification of the Sickle Cell Anemia Mutation by Means of Allele-Specific Polymerase Chain Reaction.” Human Mutation, 1:417-419.(1992)

5. Blouin, M. J., Beauchemin, H., Wright, A., De Paepe, M., Sorette, M., Bleau, A. M., Nakamoto, B., Ching-Nan, O., Stamatoyannopoulos, G., and Trudel, M. “Genetic Correction Of Sickle Cell Disease: Insights Using Transgenic Mouse Models.” Nat. Med., 6:177-182. (2000)

6. Cavard, C., et al Nucleic Acids Research, March 25;16(5):2099-110. (1988)

7. Christianson, S. W., Grenier, D. L., Hesselton, R., Leif, J. H., Wagar, E. J., Schweitzer, I. B., Rajan, T. V., Gott, B., Roopenian, D.C., and Shultz, L. D. “Enhanced Human CD4+ T Cell Engraftment In Beta Two Microglobulin-Deficient NOD-Scid Mice.” J. of Immunol., 158:3578-3586. (1997)

8. Cole-Strauss, A., Gamper, H., Holloman, W. K., Munoz, M., Cheng, N., Kmiec, E. B., Nucleic Acids Res., 27:1323-1330. (1999)

9. Cole-Strauss, A., Yoon, K., Xiang, Y., Byme, B. C., Rice, M. C., Gryn, J., Holloman, W. K., Kmiec, E. B. “Correction of the Mutation Responsible for Sickle Cell Anemia by an RNA-DNA Oligonucleotide.” (1996), Science, 273:1386. (1996)

10. Cotran, R. S., Kumar, V., Collins, T., Pathologic Basis of Disease, (6th Ed., W. B. Saunders Company, Philadelphia, Pa., 1999)

11. Doetschman, T. C., Eistetter, H., Katz, M., Schmidt, W., Kemler, R. “The In Vitro Development Of Blastocyst-Derived Embryonic Stem Cell Lines: Formation Of Visceral Yolk Sac, Blood Islands And Myocardium.” J. of Embryol. Exp. Morphol., 87:27-45. (1985)

12. Efremov, R, et al., European Journal of Biochemistry, 263:746. (1999)

13. Fritsch, G., Stimpfl, M., Kurz, M., M., Printz, D., Buchinger, P., Fischmeister, G., Hoeker, P., and Gadner, H. “The Composition of CD34 Subpopulations Differs Between Bone Marrow, Blood and Cord Blood.” Bone Marrow Transplantation, 17:169. (2000)

14. Fry, D. W., Besserer J. A. “Characterization Of Trimetrexate Transport In Human Lymphoblastoid Cells And Development Of Impaired Influx As A Mechanism Of Resistance To Lipophilic Antifolates.” Cancer Res, 48:6986. (1988)

15. Gamper, H. B. Jr., Cole-Strauss, A., Metz, R., Parekh, H., Ramesh, K., Kmiec, E. B. “A Plausible Mechanism for Gene Correction by Chimeric Oligonucleotides” Biochemistry, 39:5808-5816. (2000)

16. Gamper, H. B., Parekh, H., Rice, M. C., Bruner, M., Youkey, H., Kmiec, E. B. “The DNA strand of chimeric RNA/DNA oligonucleotides can direct gene repair/conversion activity in mammalian and plant cell-free extracts” Nucleic Acids Research, 28(21):4332-4339. (2000)

17. Goncz, K. K., Kunzelmann, K., Xu, Z., Gruenert, D. C. “Targeted Replacement of Normal and Mutant CFTR Sequences in Human Airway Epithelial Cells Using DNA Fragments.” Human Molecular Genetics, 7:1913. (1998)

18. Gluckman, E. “Umbilical Cord Blood Transplant in Human.” Bone Marrow Transplantation, 18:166 (1996)

19. Hao, Q. L., Shah, A. J. Thiemann, F. T., Smogorzewska, E. M., Crooks, G. M. “A Functional Comparison of CD34⁺CD38⁻ Cells in Cord Blood and Bone Marrow.” Blood, 86:3745. (1995)

20. Henrion, D., Amant, C., Benessiano, J., Philip, I., Plantefeve, G., Chatel, D., Hwas, U., Desmont, J. M., Durand, G., Amouyel, P., Levy, B. I. “Angiotensin, Ii Type 1 Receptro Gene Polymorphism Is Associated With An Increased Vascular Reactivity In The Human Mammary Artery In Vitro.” J. of Vascular Res., 35:356-362. (1998)

21. Inoue, N., Hirata, R. K., Russell, D. W. “High-Fidelity Correction of Mutations at Multiple Chromosomal Positions by Adeno-Associated Virus Vectors.” J. of Virol., 73:7376. (1999)

22. Jones, A T., McGavin, W J., Birch, A N E. “Effectiveness of resistance genes to the large raspberry aphid, Amphorophora idaei Borner, in different raspberry (Rubus idaeus L.) genotypes and under different environmental conditions” Annals of Applied Biology, 136(2):107-113. (2000)

23. Kelly, P., Kurtzberg, J., Vichinsky, E., Lubin, B. “Umbilical Cord Blood Stem Cells: Application for the Treatment of Patients with Hemoglobinopathies.” J. of Pediatrics, 130:695. (1997)

24. Kheradpour, A., Berman, E., Goker, E., Lin, J. T., Tong, W. P., Bertino, J. R. “A Phase II Study Of Continuous Infusion Of Trimetrexate In Patients With Refractory Acute Leukemia.” Cancer Invest, 13:36. (1995)

25. Kogler, G., Somville, T., Goebel, U., Knipper, A., Fischer, J., Adams, O., Krempe, C., McKenzie, C., Ruttgers, H., Meier, W., Bellmann, O., and Streng, H. “Haematopoietic Transplant Potential of Unrelated and Related Cord Blood: The First Six Years of the Eurocord/Netcord Bank Germany.” Klinische Padiatrie, 211:224 (1999)

26. Kren, B. T., Parashar, B. Bandyopadhyay, P., Chowdhury, J. R., Steer, C. J. “Correction of the UDP-Glucuronosyltransferase Gene Defect in the Gunn Rat Model of Crigler-Majjar Syndrome Type I with a Chimeric Oligonucleotide.” Proc. Natl. Acad. Sci. U.S.A., 96:10349. (1999)

27. Kunzelmann, K., Legendre, J. Y., Knoell, D. L., Escobar, L. C., Xu, Z., Gruenert, D. C. “Gene Targeting of CF Epithelial Cells.” Gene Therapy, 3:859. (1996)

28. Lacerda, J. F., Goker, E., Kheradpour, A., Dennig, D., Elisseyeff, Y., Jagiello, C., O'Reilly, R. J., Bertino, J. R. “Selective Treatment Of SCID Mice Bearing Methotrexate-Transport-Resistant Human Acute Lymphoblastic Leukemia Tumors With Trimetrexate And Leucovorin Protection.” Blood, 85:2675. (1995)

29. Lei, D. C., “Episomal Expression of Wild-type CFTR Corrects cAMP-dependent Chloride Transport in Repiratory Epithelial Cells.” Gene Therapy, 3:427-436. (1996)

30. Lemmer, E. R., et al., Journal of Hepatology, September 29(3):450-4. (1998)

31. Lu, L., Shen, R. N., Broxmeyer, H. E. “Stem Cells from Bone Marrow, Umbilical Cord Blood and Peripheral Blood for Clinical application: Current Status and Future Application.” Critical Reviews in Oncology-Hematology, 22:61. (1996)

32. Maddalena, A., et al., J. of Clinical Investigation, October; 82: 1353-8. (1988)

33. Mansour, S. L., Thomas, K. R., Deng, C. X., Capecchi, M. R. “Introduction Of A LacZ Reporter Gene Into The Mouse Int-2 Locus By Homologous Recombination.” Proc. Natl. Acad. Sci. U.S.A., 87:7688-7692. (1990)

34. Matsuura, K. S., et al., Am. J. of Medical Genetics, March 17;69:177-81. (1997)

35. Mistrik, I., Pavlovkin, J., Wachter, R., Pradel, K. S., Schwalm, K., Hartung, W., Mathesius, U., Stohr, C., Ullrich, C. I., “Impact of Agrobacterium tumefaciens-induced stem tumors on NO3-uptake in Ricinus communis”, Plant & Soil, 226(1):8798. (2000)

36. Nagy, A., Rossant, J., Nagy, R., Abramow-Newerly, W., Roder, J. C. “Derivation Of Completely Cell Culture-Derived Mice From Early-Passage Embryonic Stem Cells.” Proc. Natl. Acad. Sci. U.S.A., 90:8424-8428. (1993)

37. Paszty, C., Brion, C. M., Manci, E., Witkowska, H. E., Stevens, M. E., Mohandas, N., and Rubin, E. M. “Transgenic Knockout Mice With Exclusively Human Sickle Hemoglobin And Sickle Cell Disease.” Science, 278:876-878. (1997)

38. Petersen, B. E., et al. Science, 284:1168-70, 14 May 1999.

39. Piacibello, W., Sanavio, F., Garetto, L., Severino, A., Bergandi, D., Ferrario, J., Fagiolo, F., Berger, M. Aglietta, M. “Extensive Amplification And Self-Renewal Of Human Primitive Hematopoietic Stem Cells From Cord Blood.” Blood, 89:2644. (1997)

40. Russell, D. W., Hirata, R. K. “Human Gene Targeting By Viral Vectors.” Nature Genetics, 18:325. (1998)

41. Ryan, T. M., Ciavatta, D. J., and Townes, T. M. “Knockout Transgenic Mouse Model Of Sickle Cell Disease.” Science, 278:873-876. (1997)

42. Simpson, E. M., Linder, C. C., Sargent, E. E., Davisson, M. T., Mobraaten, L. E., Sharp, J. J. “Genetic Variation Among 129 Substrains And Its Importance For Targeted Mutagenesis In Mice.” Nat. Genet., 16:19-27. (1997)

43. Spencer, H. T., Sleep, S. H. E., Rehg, J. E., Blakly, R. L., Sorrentino, B. P. “A Gene Transfer Strategy For Making Bone Marrow Cells Resistant To Trimetrexate.” Blood, 87:2579. (1996)

44. Steen, V. M., Lovlie, R., MacEwan, T., McCreadie, R. G. “Dopamine D3-Receptor Gene Variant And Susceptibility To Tardive Dyskinesia In Schizophrenic Patients.” Molecular Psychiatry, 2:139-45. (1997)

45. Theilgaard-Monch, K., Raaschou-Jensen, K., Heilmann, C., Andersen, H., Bock, J., Russel, C. A., Vindelov, L., Jacobsen, N., Dickmeiss, E. “A Comparative Study of CD34+ Cells, CD34+ Subsets, Colony Forming Cells and Cobblestone Area Forming Cells in Cord Blood and Bone Marrow Allografts.” European J. of Haemotol., 62:174. (1999)

46. Wang, J. C., Doedens, M., and Dick, J. E. “Primitive Human Hematopoietic Cells are Enriched in Cord Blood Compared with Adult Bone Marrow or Mobilized Peripheral Blood as Measured by the Quantitative in Vivo SCID-repopulating Cell Assay.” Blood, 89:3919. (1997)

47. Weissman, I. L. “Translating Stem and Progenitor Cell Biology to the Clinic: Barriers and Opportunities.” Science, 287:1442. (2000)

48. Will, A. M. Archives of Disease in Childhood, 80:3. (1999)

49. Witte, R. S., Elson, P., Khandakar, J., Trump, D. L. “An Eastern Cooperative Oncology Group Phase II Trial Of Trimetrexate In The Treatment Of Advanced Urothelial Carcinoma.” Cancer, 73:688. (1994)

50. Xiang, Y., Cole-Strauss, A., Yoon, K., Gryn, J., Kmiec, E. B. “Targeted Gene Conversion in a Mammalian CD34+ Enriched Cell Population Using a Chimeric RNA/DNA Oligonucleotide.” J. of Molecular Med., 75:829. (1997)

51. Zhao ZY, Cai, TS., Tagliani, L., Miller, M., Wang, N. Pang, H., Rudert, M., Schroeder, S., Hondred, D., Seltzer, J., Pierce, D., “Agrobacterium-mediated sorghum transformation”, Plant Molecular Biology, 44(6):789-798. (2000)

52. Zhu, T., Peterson, D. J., Tagliani, L., St. Clair, G., Boszczynski., C., Bowen, B., Proc. Natl. Acad. Sci. USA, 96:8774-8778.

53. U.S. Pat. No. 6,010,908, Gruenert, Dieter C., “Gene Therapy Small Fragment Homologous Replacement.” (Jan. 4, 2000)

54. U.S. application Ser. No. 09/336,655, Davis, B. R., “Method and Device for Microinjection of Macromolecules into Non-Adherent Cell.” (filed Jun. 18, 1999(

55. www3.ncvi.nlm.nih.gov/HTBIN-post/omim/dispmim?141900

56. Gen. Bank Accession No. P19885.

We claim:

1. A non-viral gene modification method for incorporating a single stranded end-capped oligonucleotide with improved incorpoation efficiency into a target population of cells comprising:
a. preparing a formulation comprising a single stranded end-capped oligonucleotide having a nucleic acid sequence of interest corresponding to a sequence of interest to be modified in a target population of cells, wherein said sequence of interest to be modified comprises 1 to 50 bases;
b. immobilizing the target population of cells onto a substrate to provide an adherent target population of cells;
c. incorporating the single stranded end-capped oligonucleotide into the target population of cells to provide genetically modified cells; and
d. detaching said population of genetically modified cells from said substrate,
wherein greater than about 0.0001% of the target population of cells does not include the unmodified nucleic acid sequence.

2. The method of claim 1 wherein the nucleic acid sequence of interest comprises a wild-type nucleic acid sequence corresponding to an identified mutated nucleotide base or nucleotide bases of the target population of cells.

3. The method of claim 1 wherein the nucleic acid sequence of interest encodes β-globin.

4. The method of claim 1 wherein said single stranded end-capped oligonucleotide is defined at SEQ ID: No. 1.

5. The method of claim 4 wherein the end caps of the small single stranded oligonucleotides include, but are not limited to, phosphorothioate linkages between nucleotides, a backbone of methylphosphonate, phosphoramididate, morpholino peptide linkages, or nucleotides containing different 2′-halo, 2′-alkyl, or 2′-alkoxylalklyl sugars.

6. The method of claim 4 wherein the single-stranded end-capped oligonucleotide molecule has a sequence length of between 10 to 200 nucleotide bases.

7. The method of claim 1 wherein said formulation further comprises macromolecules, foreign materials, or exogenous molecules that enhance the efficiency and/or efficacy of gene modification.

8. The method of claim 7 wherein said macromolecule is a protein, a peptide fragment of said protein, a recombinant portion of said protein, or a combination thereof.

9. A cell population comprising an enriched population of genetically modified cells having a targeted gene modification artificially created using a single stranded end-capped oligonucleotide, wherein said targeted gene modification comprises a sequence of 10 to 200 nucleotides.

10. The cell population of claim 9 wherein said genetically modified cells include primary cell types, a transformed cell line, a non-transformed cell line, or a combination thereof.

11. The cell culture of claim 10 wherein said genetically modified cells normally exist in a suspended/non-adherent state.

12. The cell culture of claim 10 wherein said genetically modified cells are mammalian somatic cells.

13. The cell culture of claim 10 wherein said genetically modified cells are plant cells.

14. A transgenic animal having a site-specific genetic modification, wherein said genetic modification is provided using a single stranded end-capped oligonucleotide molecule, wherein said molecule comprises a sequence of between 10 and 200 nucleotide bases.

15. The transgenic animal of claim 14, wherein the site-specific genetic modification comprises a modification in mouse embryonic stem (ES) cells or similar cells genetically modified.

16. The non-viral gene modification method of claim 1 wherein said target cell population are somatic human hematopoietic stem/progenitor cells.

17. The non-viral gene modification method of claim 1 wherein said target population of cells are somatic human stem cells further defined as precursors of liver, pancreatic, mesenchymal, endothelial, muscle, or neuronal cells.

18. The non-viral gene modification method of claim 1 wherein the formulation is incorporated into the adherent target population of cells by needle-mediated microinjection.

19. The non-viral gene modification method of claim 1 wherein the formulation comprising the single stranded end-capped oligonucleotide is incorporated into the adherent target population of cells by electroporation, dendrimers, cationic liposome-mediated transfection, particle bombardment, iontophoresis, peptide-mediated nucleic acid delivery, red blood cell-mediated transfection, hypotonic swelling, micropricking, laser mediated introduction including the laser scissor method, and addition of the nucleic acid molecules directly to the medium surrounding the cells.

20. The non-viral gene modification method of claim 1 wherein the formulation incorporated into the cells comprises accessory oligonucleotides that enhance the efficiency and/or efficacy of gene modification.

21. The non-viral gene modification method of claim 1 further defined as a gene repair method wherein the target population of cells comprises mutant cells having an identifiable genetic mutation of from 1 to 200 bases relative to a wild-type sequence.

22. The non-viral gene modification method of claim 21 wherein the mutant cells include a mutated nucleic acid sequence with 1 to 50 mutant bases relative to a wild-type sequence.

23. The non-viral gene modification method of claim 22 wherein the mutated nucleic acid sequence comprises 1 to 20 mutant bases relative to the wild-type sequence.

24. The non-viral gene modification method of claim 23 wherein the mutated nucleic acid sequence comprises 1 to 10 mutant bases relative to the wild-type sequence.

25. The non-viral gene modification method of claim 24 wherein the mutated nucleic acid sequence comprises 1 to 5 mutant bases relative to the wild-type sequence.

26. The non-viral gene modification method of claim 25 wherein the mutated nucleic acid sequence comprises 2 mutant bases relative to the wild-type sequence.

27. The non-viral gene modification method of claim 26 wherein the mutated nucleic acid sequence is a single point mutation relative to the wild-type sequence.

28. The non-viral gene modification method of claim 21 wherein the mutated nucleic acid sequence of said target population of cells comprises the sequence at Gen. Bank Accession No. P19885.

29. A non-viral mediated method for the incorporation of a single stranded end-capped oligonucleotide with improved incorporation efficiency into a target population of cells comprising:
a. preparing a formulation comprising a single stranded end-capped oligonucleotide having a nucleic acid sequence of interest corresponding to a mutated sequence counterpart of a wild-type target gene of interest, wherein said mutated sequence comprises 1 to 50 mutant bases relative to a wild-type sequence;
b. immobilizing the population of wild-type cells to a substrate to provide adherent target cells;
c. incorporating the single stranded end-capped oligonucleotide into the adherent wild-type target cells to provide a population of genetically modified target cells comprising a mutant nucleic acid sequence; and
d. detaching said population of genetically modified cells from said substrate, wherein modified cell population comprises mutant cells that do not include the wild-type nucleic acid sequence corresponding to the mutated nucleic acid sequence, or mutant cells containing one allele with the mutant sequence and one allel with the wild type sequence.

30. The non-viral mediated method of claim 29 wherein the mutated nucleic acid sequence comprises 1 to 100 mutant nucleotides relative to the wild-type sequence.

31. The non-viral mediated method of claim 29 wherein the mutated nucleic acid sequence comprises 1 to 50 mutant nucleotides relative to the wild-type nucleic acid sequence.

32. The non-viral mediated method of claim 29 wherein the mutated nucleic acid sequence comprises 1 to 20 mutant nucleotides relative to the wild-type sequence.

33. The non-viral mediated method of claim 29 wherein the mutated nucleic acid sequence comprises 1 to 10 mutant nucleotides relative to the wild-type sequence.

34. The non-viral mediated method of claim 29 wherein the mutated nucleic acid sequence comprises 1 to 5 mutant nucleotides relative to the wild-type sequence.

35. The non-viral mediated method of claim 29 wherein the mutated nucleic acid sequence comprises 2 mutant nucleotides relative to the wild-type sequence.

36. The non-viral mediated method of claim 29 wherein the mutated nucleic acid sequence comprises 1 mutant nucleotide relative to the wild-type sequence.

37. The non-viral mediated method of claim 29 wherein the target population of cells comprises primary cells types, transformed, or non-transformed mammalian somatic cells.

38. The non-viral mediated method of claim 29 wherein said modified cells exist in a suspended/non-adherent state.

39. The non-viral mediated method of claim 37 wherein the target population of cells comprise endothelial cells.

40. The non-viral mediated method of claim 37 wherein the target population of cells comprise somatic stem cells including, but not limited to, hepatic, neuronal, endothelial, or mesenchymal stem cells.

41. The non-viral mediated method of claim 37 wherein the target population of cells comprise murine (mouse) embryonic stem cells.

42. The non-viral mediated method of claim 29 wherein the target population of cells comprise plant cells.

43. The method of claim 42 wherein the substrate for attachment of the target population of plant cells includes, but is not limited to, plant lectin binding carbohydrates, agarose, agar, their derivatives, or combinations thereof.
